(19) United States
(12) Patent Application Publication
(10) Pub. No.: US 2009/0271892 A1
(43) Pub. Date: Oct. 29, 2009

(54) LYSOPHOSPHATIDIC ACID ACYLTRANSFERASE GENES AND USES THEREOF

(76) Inventors: Brigitte Thomasset, Compiegne (FR); Emma Wallington, Cambridge (GB); Jeroen Wilmer, Levignac (FR); Eric Gontier, Germiny (FR); Sebastien Gougeon, Porcelette (FR)

Correspondence Address: CONNOLLY BOVE LODGE & HUTZ, LLP, P O BOX 2207, WILMINGTON, DE, 19899, US

(21) Appl. No.: 12/303,484

(22) PCT Filed: Jun. 5, 2007
(86) PCT No.: PCT/EP2007/055502

§ 371 Date: Jun. 26, 2009

(30) Foreign Application Priority Data
Jun. 6, 2006 (EP) 06114983.7

(51) Int. Cl. C12N 15/82; C12N 15/54; C12N 5/10; A01H 5/00; C12N 9/10; C07K 14/415

(52) U.S. Cl. 800/281; 536/23.2; 435/320.1; 435/468; 435/419; 800/298; 435/193; 530/377

(57) ABSTRACT

The present invention relates to the identification and characterization of new lysophosphatidic acid acyltransferases (LPAAT) as well as to the use of these enzymes for modifying plants for efficient production of modified lipids.

### FIELD OF INVENTION

The invention relates to the efficient production and storage of cyclic fatty acids in plants. The production process particularly uses genetically modified plants.

### BACKGROUND

Plant oils have a wide range of compositions. The constituent fatty acids determine the chemical and physico-chemical properties of the oil which in turn determine the utility of the oil. Plant oils are used in food and increasingly in non-food industrial applications, particularly lubricants.

To reduce environmental impact, the production of efficient biodegradable lubricants has been contemplated. The starting materials for such lubricants are plant oils.

Classical plant oils from crops grown on a commercial scale typically contain saturated and unsaturated linear fatty acids with chain lengths between 12 and 18 carbon atoms. The physical properties of these fatty acids do not meet the requirements for high-performance lubricants.

To obtain a sufficient lubricant function, the carbon chains need to be long enough, probably around 16 to 18 carbon atoms. With saturated chains of this length the melting point and cloud point increase to unacceptable levels for use in car engines.

With the requirement for long chains, modifications of the saturated chain are required that reduce the melting point. In classical plant oils these modifications are desaturations, which lead to the desired properties as a lubricant. However, unsaturated fatty acids have an additional problem, in that they are oxidatively unstable, and therefore have a short functional life.

To address these problems, it has been shown that it is particularly advantageous to use branched chain fatty acids as a lubricant base (WO 99/18217). The synthetic route selected is the production of the intermediate cyclopropane fatty acids in plant cells for conversion into branched chain fatty acids by industrial processing.

Cyclic fatty acids containing three carbon carbocyclic rings, especially cyclopropane fatty acids, are of particular industrial interest. The cyclopropane fatty acids have physical characteristics somewhere between saturated and monounsaturated fatty acids. The strained bond angles of the carbocyclic ring are responsible for their unique chemistry and physical properties. Hydrogenation allows the ring to open with the production of methyl-branched fatty acids. These branched fatty acids have the low temperature properties of unsaturated fatty acids and their esters without susceptibility to oxidation. Such branched fatty acids are therefore eminently suitable for use in lubricants.

Further they may be used as a replacement for “isostearate” a commodity in the oleochemical industry which is included in the formulation of cosmetics and lubricant additives, for example. The highly reactive nature of the strained ring also encourages a diverse range of chemical interactions allowing the production of numerous novel oleochemical derivatives.

Broadly speaking, there are two main approaches to altering the lipid composition of an oil, which to date have been applied as alternatives. Firstly, plants may be modified to produce fatty acids which are foreign to the native plant. For example, rape may be modified to produce laureate which is not naturally produced by that plant. Secondly, the pattern and/or extent of incorporation of fatty acids into the glycerol backbone of a lipid may be altered.

Lipids are formed by the addition of the fatty acid moieties into the glycerol backbone by acyltransferase enzymes. There are three positions on the glycerol backbone at which fatty acids may be introduced. The acyltransferase enzymes which are specific for each position are hence referred to as 1-, 2-, and 3-acyltransferase enzymes respectively or more precisely glycerol-3-phosphate acyltransferase (GPAT), lysophosphatidic acid transferase (LPAAT) and diacyl-glycerol acyltransferase (DAGAT), Ohlrogge and Browse 1995, The Plant Cell 7: 957-970.

It is most interesting to use 2-acyltransferases, incorporating the fatty acid at the sn-2 position of the glycerol, since this category of acyltransferase shows higher fatty acid specificity than either 1-acyltransferases or 3-acyltransferases. It is interesting to note that different types of such 2-acyltransferases can occur in plants.

Constitutive 2-acyltransferases (also called “type 1”) are found in every cell of plants, and their fatty acid substrates will eventually finish within the cell membranes. Seed-specific 2-acyltransferases (also called “type 2”) are expressed in seed, and will actually be used for storage of unusual fatty acids produced in the seed. Such type 2 acyltransferases have been identified, for example from Limnanthes, or from coconut. It is quite surprising that no such type 2 acyltransferase is currently known in rape, while this plant stores very long chain fatty acids (vLCFA) in its seeds. In the present application, and unless specifically indicated, foreseen acyltransferases will be sn-2 acyltransferases, incorporating fatty acids at the sn-2 position of the glycerol backbone, only their type (as indicated above) will be specified.

It has previously been demonstrated that it is possible to introduce cyclic fatty acid synthase (CFAS) genes into plant cells and in this way produce cyclic fatty acids in plant cells. In fact, cyclic fatty acids (especially cyclopropane fatty acids) are rather unusual in plants and although as early as 1978 and 1980, respectively, cyclopropenes and cyclopropanes had been identified in few plant seeds, their biochemical synthesis has not been elucidated.

Recently CFAS have been identified and characterized in Sterculia foetida (WO 03/060079) and in lychee (WO 2006/087364). The protein sequences are described respectively as SEQ ID No 8 and 10 and SEQ ID No 6 and 7.

The genes coding for these proteins have successfully been proved to be able to produce cyclopropane in various organisms such as E. coli or plants. It is obviously interesting to produce these cyclic fatty acids in plants, but necessary to be able to properly store them within the glycerolipids, in order to make an efficient system of production. It is thus very interesting to be able to produce a transgenic plant that would contain cyclic fatty acid synthase, such as the ones disclosed above, as well as a LPAAT that would use these as substrates.

The inventors have now identified in Lychee a nucleic acid sequence that codes for a protein that has LPA acyltransferase activity. Surprisingly, a mutant of this protein, in the C-terminal part, also presents such activity.

These nucleic acid sequences can thus be very useful for the efficient incorporation of cyclopropane fatty acids into glycerol lipids in plants, in particular in the seeds of especially high oil-producing crop plants.

Furthermore, it is interesting to note that this protein has specificity for unusual fatty acids, a type 2-like activity, while it presents homology to type 1-acyltransferases.

### SUMMARY OF THE INVENTION

The present invention relates to the identification and characterization of a plant cyclopropane-incorporating LPAAT and the identification and cloning of the relevant gene sequence. The invention also relates to the use of that gene for the efficient production of cyclopropane fatty acids in an oilseed crop.

The invention specifically relates to a LPAAT from a plant in which the major cyclic fatty acids accumulated in the seed are cyclopropane fatty acids.

### FIGURE

FIG. 1: Kinetics of phosphatidic acid synthesis using ¹⁴C-C18:1-CoA on ³H-LPA as substrates and Brassica napus (SEQ ID No 5) and Litchi microsomal (SEQ ID NO 1) LPAATs as enzymes. Duplicate measurements for one experiment are shown.

FIGS. 2 to 6: plasmids pEWX6, pEWX4, pEW80-SCV, pEW88-SCV and pEWX8 used for transformation of rapeseed.

### DESCRIPTION

One aspect of the invention relates to isolated nucleic acids encoding a lysophosphatidic acid acyltransferase (LPAAT).

In a specific embodiment, said LPAAT is isolated from a plant, in particular from the family of Sapindaceae.

The Sapindaceae are members of an interesting family mainly found in the tropics. The only two plants identified to date that have seeds in which cyclopropane fatty acids accumulate without any cyclopropene fatty acids belong to this family. Litchi sinensis (Lychee) and Euphoria longana (Longan) are both eaten as tropical fruits and do not have seeds with a high oil content. It is believed that they contain acyltransferases with a specific activity, which may be different from the one seen in other oil plants such as rape.

In a specific embodiment, the invention relates to an isolated nucleic acid encoding a protein having LPA acyltransferase activity, wherein said protein comprises:
a. a sequence encoding the amino acid sequence set forth in SEQ ID No 1.
b. a sequence that is at least 90%, 95%, 97%, 98%, 99% identical to the sequence in a., wherein said sequence codes for a protein having acyltransferase activity
c. a fragment of the sequence in a or b, wherein said fragment contains at least 350 amino acids and codes for a protein having acyltransferase activity.

In the preferred embodiment, the protein coded by said isolated nucleic acid harbors LPAAT activity, when introduced into E. coli or in a plant, especially oilseed rape or linseed, according to the method described in the examples.

The inventors have demonstrated that it is possible to isolate a nucleic acid coding for a LPAAT from Lychee, but also variations in the C-terminus end of this protein lead to functional LPAAT.

The invention thus also relates to the variant of the Lychee LPAAT depicted in SEQ ID No 2, in particular in its last 6 amino acids, which also retains LPAAT activity when tested according to the examples. Mutants of the protein are obtained by insertion/deletion/replacement of amino acids of said protein. Obtaining and testing said mutants is well within the skills of the person in the art, using for example well described targeted mutagenesis techniques and the teachings of the examples.

As a preferred embodiment, the invention relates to an isolated nucleic acid that encodes a protein comprising SEQ ID No 2, and preferably consisting of SEQ ID No 2.

Two polynucleotides or polypeptides are said to be “identical” if the sequence of nucleotides or amino acid residues, respectively, in the two sequences is the same when aligned for maximum correspondence.

Sequence comparisons between two (or more) polynucleotides or polypeptides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math 2: 482 (1981), by the homology alignment algorithm of Neddleman and Wunsch, J. Mol. Biol. 48: 443 (1970), by the search for similarity method of Pearson and Lipman, Proc. Natl. Acad. Sci. (U.S.A.) 85: 2444 (1988), by computerized implementation of these algorithms (GAP, BESTFIT, BLAST N, BLAST P, FASTA, and TFASTA in the Wisconsin Genetics Software Package, Genetics Computer Group (GCG), 575 Science Dr., Madison, Wis.), or by inspection.

Preferably, the percentage of identity of two polypeptides is obtained by performing a blastp analysis with the sequence encoded by the nucleic acid according to the invention, and SEQ ID No 1, using the BLOSUM62 matrix, with gap costs of 11 (existence) and 1 (extension), or by the Needleman and Wunsch method.

The percentage of identity of two nucleic acids is obtained using the blastn software, with the default parameters as found on the NCBI web site (http://www.ncbi.nlm.nih.gov/BLAST/), or using the Needleman and Wunsch method.

“Percentage of sequence identity” is also determined by comparing two optimally aligned sequences over a comparison window, where the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

In another embodiment, the invention relates to an isolated nucleic acid comprising a sequence that is greater than 80%, preferably greater that 90%, more preferably greater than 95%, more preferably greater than 97, 98 or 99% identical to any of SEQ ID No 3 or SEQ ID No 4 and that codes for a protein having LPAAT activity.

In a preferred embodiment, said isolated nucleic acid comes from Litchi sinensis or a plant of the family of Sapindaceae.

More preferably said nucleic acid comprises nucleotides 1-1161 of SEQ ID NO 3 or SEQ ID No 4. The invention also encompasses a nucleotide sequence that is a fragment of SEQ ID No 3 or SEQ ID No 4 and that codes for a LPAAT.

The two LPAAT proteins depicted in SEQ ID No 1 and SEQ ID No 2 have homology to previously identified plant LPAAT, from type 1. It is nevertheless surprising to see that their activity is more of a type 2 LPAAT than of type 1, regarding their specificity to use unusual substrates.

Another aspect of the invention relates to a chimeric gene comprising a nucleic acid sequence according to the invention operatively linked to suitable regulatory sequences for functional expression in plants, and in particular in the seeds of oil plants. The phrase “operatively linked” means that the specified elements of the component chimeric gene are linked to one another in such a way that they function as a unit to allow expression of the coding sequence. By way of example, a promoter is said to be linked to a coding sequence in an operational fashion if it is capable of promoting the expression of said coding sequence. A chimeric gene according to the invention can be assembled from the various components using techniques which are familiar to those skilled in the art, notably methods such as those described in Sambrook et al. (1989, Molecular Cloning, A Laboratory Manual, Nolan C., ed., New York: Cold Spring Harbor Laboratory Press). Exactly which regulatory elements are to be included in the chimeric gene will depend on the plant and the type of tissue in which they are to work: those skilled in the art are able to select which regulatory elements are going to work in a given plant.

In order to produce a significant quantity of the protein according to the invention in plant tissues it is much preferable to drive the expression of the newly identified LPAAT genes with a suitable plant promoter. Many promoters are known and include constitutive and tissue and temporally specific.

For expressing the protein in another organism, such as a microorganism or another eukaryotic cell, suitable promoters are well known in the art.

Promoter sequences of genes which are expressed naturally in plants can be of plant, bacterial or viral origin. Suitable constitutive promoters include but are not restricted to octopine synthase (Ellis et al, 1987, EMBO J. 6, 11-16; EMBO J. 6, 3203-3208), nopaline synthase (Bevan et al, Nucleic Acids Res. 1983 Jan. 25; 11(2):369-85), mannopine synthase (Langridge et al, PNAS, 1989, vol. 86, 9, 3219-3223) derived from the T-DNA of Agrobacterium tumefaciens; CaMV35S (Odell et al, Nature. 1985 Feb. 28-Mar. 6; 313(6005):810-2) and CaMV19S (Lawton et al Plant Mol. Biol. 9:315-324, 1987) from Cauliflower Mosaic Virus; rice actin (McElroy et al, Plant Cell, 2:163-171, 1990), maize ubiquitin (Christensen et al, 1992, Plant Mol Biol 18: 675-689) and histone promoters (Brignon et al, Plant J. 1993 September; 4(3):445-57) from plant species. Sunflower ubiquitin promoter is also a suitable constitutive promoter, Binet et al., 1991, Plant Science, 79, pp 87-94).

It is preferable that the LPAAT genes are expressed at a high level in an oil producing tissue to avoid any adverse effects of expression in plant tissues not involved in oil biosynthesis and also to avoid the waste of plant resources; commonly the major oil producing organ is the seed.

Thus, in a preferred embodiment, the chimeric gene of the invention comprises a seed specific promoter operatively linked to the nucleic acid of the invention. Suitable promoters include but are not limited to the most well characterised phaseolin (Sengupta-Gopalan et al., 1985, Proc Natl Acad Sci USA 85: 3320-3324), conglycinin (Beachy et al., 1985, EMBO J. 4: 3407-3053), conlinin (Truksa et al, 2003, Plant Phys and Biochem 41: 141-147), oleosin (Plant et al., 1994, Plant Mol Biol 25(2): 193-205), and helianthinin (Nunberg et al., 1984, Plant Cell 6: 473-486).

In a very preferred embodiment, said promoter is the Brassica napus napin promoter (EP 255278), being seed specific and having an expression profile compatible with oil synthesis.

In another very preferred embodiment, said promoter is from a FAE1 (Fatty acid Elongase1; W02/052024).

The invention also relates to a transformation vector, in particular a plant transformation vector comprising a nucleic acid molecule or a chimeric gene according to the invention. For direct gene transfer techniques, where the nucleic acid sequence or chimeric gene is introduced directly into a plant cell, a simple bacterial cloning vector such as pUC19 is suitable. Alternatively more complex vectors may be used in conjunction with Agrobacterium-mediated processes. Suitable vectors are derived from Agrobacterium tumefaciens or rhizogenes plasmids or incorporate essential elements from such plasmids. Agrobacterium vectors may be of co-integrate (EP 116718) or binary type (EP 120516). These methods are well known in the art.

The invention also relates to a method for expressing a LPAAT protein in a host cell, in particular a plant cell comprising transforming said cell with an appropriate transformation vector according to the invention. In the case of a plant cell, one would be transfecting a suitable plant tissue with a plant transformation vector. Integration of a nucleic acid or chimeric gene within a plant cell is performed using methods known to those skilled in the art. Routine transformation methods include Agrobacterium-mediated procedures (Horsch et al, 1985, Science 227:1229-1231). Alternative gene transfer and transformation methods include protoplast transformation through calcium, polyethylene glycol or electroporation mediated uptake of naked DNA. Additional methods include introduction of DNA into intact cells or regenerable tissues by microinjection, silicon carbide fibres or most widely, microprojectile bombardment. All these methods are now well known in the art.

A whole plant can be regenerated from a plant cell. A further aspect relates to a method for expressing a LPAAT protein in a plant comprising transfecting a suitable plant tissue with a plant transformation vector and regeneration of an intact fully fertile plant. Methods that combine transfection and regeneration of stably transformed plants are well known.

Thus a further aspect of the invention relates to a plant transformed with a gene coding for a LPAAT according to the invention. Any plant that can be transformed and regenerated can be included. An embodiment relates to a plant where the original plant is an oil producing crop plant. Preferred plants include the oilseed crops such as rape, linseed, sunflower, safflower, soybean, corn, olive, sesame and peanuts. Most preferred are plants that produce oleic acid.

Transformation methods are known for sunflower such as those described in WO 95/06741 and more recently Sankara Rao and Rohini, (1999, Annals of Botany 83: 347-354).

A preferred embodiment is a plant transformed with a gene coding for a LPAAT according to the invention where the original plant is Brassica napus. This can be achieved by known methods such as Moloney et al, Plant cell reports 8: 238-242, 1989.

Another preferred embodiment is a plant transformed with a gene coding for LPAAT according to the invention where the original plant is linseed. Linseed transformation was first achieved in 1988 by Jordan and McHughen (Plant cell reports 7: 281-284) and more recently improved by Mlynarova et al (Plant Cell reports, 1994, 13: 282-285).

Another embodiment of the invention encompasses a plant according to the invention that also contains a gene coding for a cyclic fatty acid synthase, in particular coding for SEQ ID No 6 or SEQ ID No 7 or SEQ ID No 8 or SEQ ID No 10. These plants are obtained, for example, by crossing a plant as described above with a plant that contains a vector containing said gene coding for a CFAS. Another way to obtain these double transgenic plants may be to use cotransformation, with one or two vectors containing both CFAS and LPAAT coding genes. These methods are well known in the art.

Another aspect of the invention relates to the oil produced by a plant transformed with a gene coding for a LPAAT according to the invention. In particular, when the invention encompasses transformation of a plant with a LPAAT according to the invention and a CFAS, a preferred embodiment is oil having an increased proportion of cyclopropane fatty acids. A most preferred embodiment is oil having an increased proportion of dihydrosterculic acid.

### EXAMPLES

All DNA modifications and digestions were performed using enzymes according to the manufacturers' instructions and following protocols described in Sambrook and Russell, 2001; Molecular Cloning, A Laboratory Manual.

### Example 1

### Identification of Lysophosphatidic Acid-Acyl Transferases (LPAAT)

The inventors have identified one putative Lysophosphatidic Acid-Acyl Transferase from Lychee (SEQ ID No 2). They also have obtained a mutated protein derived from this protein, which is depicted as SEQ ID No 1.

Both proteins present 387 amino acids, and they are about 99.0% identical. It is interesting to note that they are 100% identical apart from the last 5 amino acids. These proteins present homology with 2-acyltransferase of type 1 from plants.

### Example 2

### Functional Validation of LPAAT in E. coli

Plasmids pEW108 and 117 comprising genes coding for SEQ ID No 1 and SEQ ID No 2 respectively under the control of the araBAD promoter (Guzman, L-M. et al. (1995) J Bacteriology 177: 4121-4130) were prepared and used to transform and complement an E. coli strain. The araBAD promoter is induced by arabinose, repressed by glucose and is used in the art to express polynucleotides in a controlled manner.

### i) Acyl-CoA Synthesis

As C19CA-CoA is not commercially available it has been synthesized using the enzymatic method of Taylor et al. (1990 Analytical Biochem., 184, 311-316). C19CA has been purchased from Larodan AB (ref. 13-1909-7) and yeast coenzyme-A (ref. C-3144) and yeast EC 6.2.1.1 S-acetyl-coenzyme-A synthetase was purchased from Sigma (ref. A-1765, S. cerevisiae). Two milligrams of C19CA are added to a buffer containing the following components at the final concentrations indicated: Triton X-100 (0.1% w/v), CoA (5 mM), dithiothreitol (DTT, 1 mM), ATP (10 mM), MgCl₂ (10 mM) and 3-N-morpholinopropanesulfonic acid (Mops)-NaOH (100 mM, pH7.5) and flushed with nitrogen. The mix was sonicated for 10 min in an ultrasonic bath in order to emulsify the C19CA then Acyl-CoA synthetase (1.45 units) is added. The 4 mL final volume was then incubated at 35° C. for 2 h.

After incubation, the reaction mixture was directly applied to a disposable Prep-Sep C₁₈ extraction column (Alltech ref. 205000U) previously washed with 5 mL of HPLC-grade methanol and equilibrated with 5 mL of 100 mM Mops-NaOH, pH7.5. After the 4 mL sample application, the column was washed with 5 mL of 100 mM Mops-NaOH, pH7.5. Then, C19CA-CoA was eluted with 20 mL methanol. The solvent was evaporated under reduced pressure in a Rotavapor (Labo-Rota S-300, Resona Technics) and the residue was redissolved in 5 mL Na-acetate buffer (100 mM, pH5); flushed with nitrogen and kept at −18° C.

Concentration of C19CA-CoA was determined by OD measurement at 254 nm and in comparison with C18:1-CoA standard absorption curve.

ii) Transformation and Complementation of E. coli

The following experiments were performed with the Escherichia coli JC201 mutant strain, which is temperature conditional in endogenous LPAAT activity and able to grow at 30° C. but not at 44° C. (genotype plsC, described in Coleman, 1990 J. Biol. Chem., 265 (28), 17215-21.). These bacteria were kept at −80° C. as 1 ml glycerol stocks. For E. coli JC201 transformation, one glycerol stock was diluted in 15 mL of Luria-Bertani (ref. L3022, Sigma) liquid medium and cultivated overnight at 30° C. Then, 1 mL was used to inoculate flasks containing 15 mL of fresh liquid LB medium. Optical density was measured at 600 nm and each flask was then cultivated for 6 hours at 30 and 44° C. respectively in order to check by optical density measurement that bacterial growth occurs at 30° C. but not at 44° C. One of the cultures obtained at 30° C. was then centrifuged at 4500 g/4° C. for 10 min. Supernatant was discarded and the pellet was kept on ice for 2 hours. The pellet was washed three time with 15 mL sterile distilled water and then twice with 15 mL distilled sterile water containing 10% (weight/volume) glycerol (centrifugation condition: 4500 g/4° C./10 min). The final pellet was then resuspended in 1 mL sterile distilled water containing 10% glycerol. Fifty microliters of this suspension were then mixed with 1 μL of plasmid solution prepared in sterile distilled water at a concentration of 30 ng of dried plasmid per microliter. This mix was placed in the 2 mm cuvette of a Bio-Rad Gene Pulser Xcell (voltage: 2.5 kV; capacitance: 25 μF; resistance: 200Ω) for obtaining bacterial transformation via electroporation. Immediately after the electric pulse application, 500 μL of LB (previously kept in ice) were added in the cuvette. The total volume was then placed in an Eppendorf tube and kept at 30° C. for an hour prior to use for inoculation of a Petri dish (LB agar 100 μg mL-1 ampicillin). After 48 h at 30° C., three isolated colonies were collected and cultivated separately overnight in 15 ml of liquid RM medium (Casamino Acid: 20 g/l; Na2HPO4: 42 mM; KH2PO4: 22 mM; NaCl: 8 mM; NH4Cl: 18 mM; MgCl2: 1 mM; Thiamine: 0.1 mM) containing 100 μg mL-1 ampicillin.

In order to check that transformed E. coli cells are producing functional recombinant LPAAT, a complementation test was performed as follows.

Two hundred microliters of one of the above cultures were used to inoculate each of four flasks containing 15 mL of RM medium. Two of these flasks were complemented with arabinose 0.02% (w/v). The two others were complemented with glucose 0.02%. After OD600 nm measurement and 3 h of culture at 30° C., OD600 nm was measured again (this first culture step allows bacterial growth and LPAAT expression). Then one flask containing arabinose and one flask containing glucose were incubated at 30° C. (A30 and G30) and the two others were incubated at 44° C. (A44 and G44) for 4 hours. OD600 nm was measured again to check that growth occurred in culture A30, G30 and A44 but not in G44.

In conclusion, the cloned sequences complement the JC201 mutant indicating that both Lychee clones encode functional LPAATs.

iii) Isolation of Microsomes

The cultures A30 and A44 were pooled and cells centrifuged at 4500 g for 10 min, 4° C. The pellet was collected for microsome extraction.

Cell were resuspended in 5 ml of 50 mM Tris/HCl, pH7.5, 0.1 mM Pefabloc SC (ref 76307, Sigma) and broken through a cell disrupter (Ultrasonic Processor, amplitude: 50) at 4° C. The resulting mixture was centrifuged at 10000 g for 20 min, 4° C. The supernatant was centrifuged at 20000 g for 30 min, 4° C. and the pellet discarded before a final centrifugation at 100000 g for 3 h at 4° C. The microsomal pellet was resuspended in 1 ml of 50 mM Tris/HCl, pH7.5, 0.1 mM Pefabloc SC, and 150 μl aliquots were frozen in liquid nitrogen before storage at −80° C.

### iv) LPAAT Assay

The ³H-oleoyl-lysophosphatidic acid (³HLPA, ref. NET1100; 600 μM, 28 mCi mmol⁻¹) and ¹⁴C-Oleoyl-CoA (¹⁴CC18:1-CoA, ref. NET651A; 300 μM, 11 mCi mmol⁻¹) radio-labeled substrates were purchased from Perkin Elmer. Assays were carried out in a final volume of 300 μl and contained Tris/HCl (100 mM, pH7.5), Triton X-100 (0.01% w/v), BSA (1 mg/ml), ascorbic acid (10 mM), EDTA (2 mM), 100 μl LPA and 50 μl acyl-CoA. The reaction was started by the addition of 30 μl of microsomes, and conducted in a glass vial placed in a Eppendorf Thermomixer-compact apparatus (30° C., 350 rpm). The reaction was stopped after incubation (from 0 to 120 min) by addition of 720 μl of chloroform/methanol (1:1). To separate the phases, 280 μl of 1M KCl in 0.2 M H₃PO₄ were added and the whole mixture was vortexed for 10 s before centrifugation at 1300 g, 5 min at room temperature. The upper aqueous phase was discarded and 2×25 μl of the remaining organic phase was spotted on to silica gel 60 ÅF254 TLC plates (ref. 1.05715, Merck) and developed in chloroform/methanol/NH₄OH/water (65:25:0.9:3). The phosphatidic acid spot was visualized by iodine revelation and collected for scintillation counting.

Radioactivity measurement was performed in 10 mL Ultimagold (ref. 6013329, Perkin Elmer) using a liquid scintillation analyzer (Tri-Carb 2100TR, Packard) for determination of ³H and ¹⁴C separately.

LPAAT Assay with C18:1-CoA as Substrate:

A first LPAAT assay was performed as described above in order to determine optimal LPAAT activity using ¹⁴CC18:1-CoA as substrate:
Effect of incubation time was tested for 0, 1, 3, 5, 10, 30, 60 and 120 min with 50 μM ¹⁴CC18:1-CoA at 30° C.
In order to determine LPAAT kinetic parameters, the effect of C18:1-CoA concentration was tested as described above. Incubation time was fixed at 10 min with 0, 1, 3, 5, 10, 20 and 50 μM of ¹⁴CC18:1-CoA. For all experimental conditions, the mean of 2 values is calculated. The experiment has been performed three to five times (table 1). All the results are expressed in pmol of PA synthesized per mg of total protein and per minute.

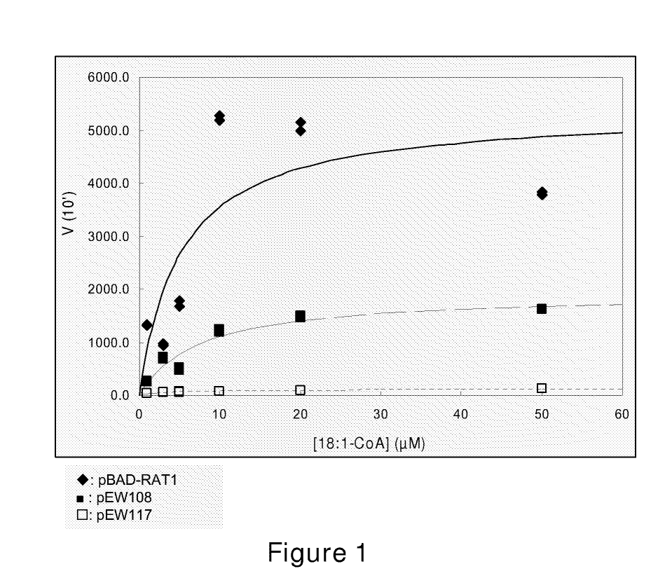

Presence of PA in the lipid fraction reveals that B. napus and L. sinensis LPAATs (BnLPAAT and LsLPAAT respectively) are expressed in the transformed E. coli mutant strains and are fully functional as they allow the esterification of ¹⁴CC18:1-CoA on ³H-LPA (FIG. 1).

The calculated values for Kₘ and apparent Vₘₐₓ demonstrate that LsLPAAT (pEW108) and BnLPAAT have similar affinity for C18:1-CoA and comparable activity, making LsLPAAT a competitive enzyme for modifying oil composition in plants (table 1).

TABLE 1
Evaluation of the BnLPAAT and LsLPAAT enzyme
kinetics with C18: 1-CoA as substrate.

| | Km | Vmax | |
|---|---|---|---|
| LPAAT | (μM) | (pmol · mg prot⁻¹ · min⁻¹) | N |
| Bn (RAT1) | 10.6 ± 6.7 | 5597 ± 2703 | 3 |
| Ls (pEW108) | 7.3 ± 4.1 | 1557 ± 616 | 4 |
| Ls (pEW117) | 3.6 ± 1.7 | 115 ± 31 | 5 |

The values are calculated from multiple experiments (N), each with 2 replicates.

### Competitive LPAAT Assay Using C18:1-CoA and C19CA-CoA:

Competitive LPAAT assays were performed as described above except that incubations were done with 25 μM ¹⁴CC18:1-CoA and 0, 10 or 25 μM C19CA-CoA for ten minutes. For all experimental conditions, data are derived from three experiments with 4 replicates each.

LPAAT selectivity for the two substrates is calculated as follows:

Selectivity for C18:1-CoA: S^{C18:1-CoA}=¹⁴C/³H

Selectivity for C19CA-CoA: S^{C19:1CA-CoA}=(³H—¹⁴C)/³H

in which, ³H and ¹⁴C are the molar quantity of produced PA and the molar quantity of ¹⁴C18:1 incorporated into this PA. These molar quantities are calculated from the corresponding ³H and ¹⁴C radioactivity levels measured in the phosphatidic acid spots scraped from TLC plates. Selectivity is expressed as the fraction of the specific fatty acid incorporated out of the total incorporated at the sn-2 position.

Competitive assays using C19CA-CoA and C18:1-CoA demonstrate that both BnLPAAT and LsLPAAT can use C19CA-CoA as substrate.

Nevertheless, the results obtained demonstrate that BnLPAAT displays a very low selectivity for this substrate, with very little incorporation of 19:0CA up to concentrations of 50 μM, well above expected physiological levels. LsLPAAT selectivity for C19CA-CoA is higher than that of BnLPAAT (table 2), resulting in up to 35% of fatty acids incorporated at the sn-2 position being 19:0CA, even at low total concentrations of acyl-CoA. This indicates that the activity of LsLPAAT is significantly different from that of BnLPAAT.

TABLE 2
Selectivity of BnLPAAT and LsLPAAT for
C19CACoA in competition with C18: 1CoA.

| | | | | Average |
|---|---|---|---|---|
| | C18: 1CoA (μM) | C19CA (μM) | Selectivity | selectivity |
| BnLPAAT | 25 | 0 | / | 0.054 ± 0.057 |
| | 25 | 10 | ≈0.00 | |
| | 25 | 25 | ≈0.00 | |
| | 50 | 25 | 0.01 | |
| | 50 | 50 | 0.19 | |
| LsLPAAT | 25 | 0 | / | 0.377 ± 0.101 |
| | 25 | 10 | 0.15 | |
| | 25 | 25 | 0.34 | |
| | 50 | 25 | 0.28 | |
| | 50 | 50 | 0.35 | |

It could be deduced from these results that LsLPAAT acts more like a type 2 acyltransferase, with its ability to use non-usual fatty acids as substrates, while BnLPAAT acts like a type 1 acyltransferase.

### Example 3

### Functional Validation of LsLPAAT in Brassica napus

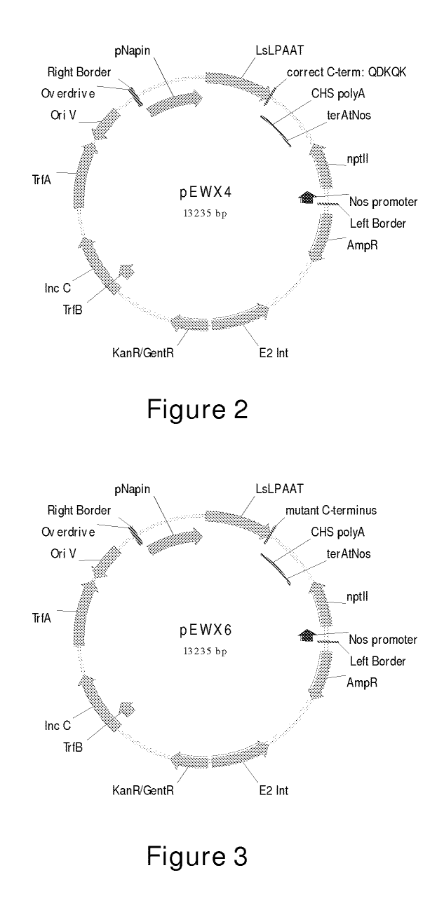

Plasmids producing SEQ ID No 1 and SEQ ID No 2 under the control of the napin promoter are created by cloning the LsLPAAT encoding region from pEW108 or pEW117 as 1165 bp NcoI-EcoRI fragments into pEntr4 NcoI-EcoRI sites to create pEWX5 and pEWX3. These are then recombined into a suitable binary vector, pNapR12-SCV, to create pEWX6 and pEWX4 respectively (FIGS. 3 and 2). The modified binary vector in turn is introduced into Agrobacterium tumefaciens strain C58pMP90.

Transgenic rape plants are produced with the A. tumefaciens carrying one or other vector according to the method of Moloney et al, 1989. Expression of the transgene is confirmed by RT-PCR after RNA is isolated from ten 30 day post anthesis seeds using RNeasy kit (Qiagen) with on-column DNase digestion following the protocol from the manufacturer. Lines with a single copy of the transgene are also identified by Q-PCR.

Transgenic lines with a single copy of the transgene and having high LsLPAAT expression are selected for crossing with rape plants transformed with an A. tumefaciens strain carrying an expression cassette encoding a cyclic fatty acid synthase (CFAS), either SEQ ID No 6, SEQ ID No 7, SEQ ID No 8 or SEQ ID No 10 under the control of a seed specific promoter, such as the napin promoter or the promoter described in WO 02/052024.

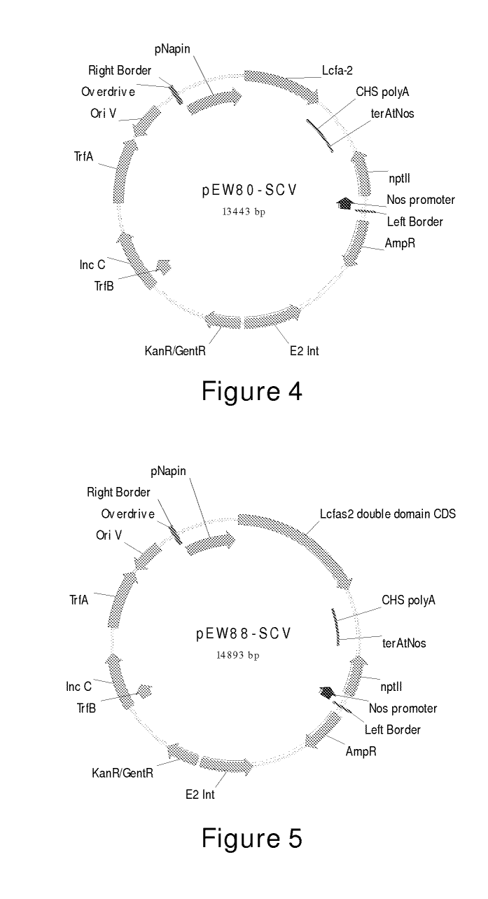

Rape plants transformed with pEW80-SCV and pEW88-SCV (FIGS. 4 and 5) producing Lychee CFAS (SEQ ID No 6 and SEQ ID No 7) have previously been described in PCT/EP2006/060030.

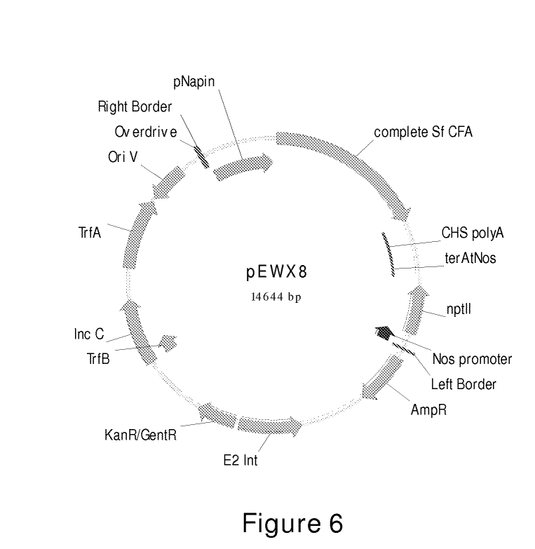

The Sterculia CFAS sequence (nucleic acid coding for SEQ ID No 8) is amplified from the 2nd codon through to the stop codon as a 2.6Kb product and is ligated into pEntr4 NcoI-EcoRV sites which have been filled in using Klenow polymerase to create pEWX7 in which the start codon is restored to the reading frame. This construct is then recombined with the binary vector pNapR12-SCV to create pEWX8 (FIG. 6). Transgenic rape plants expressing the transgene at a high level are identified by RT-PCR.

Following crossing, lipids are extracted from the immature seed collected from individual double transgenic rape plants and the fatty acids profile determined by GC. The presence of cyclic fatty acids incorporated at the Sn-2 position is demonstrated.

1. An isolated nucleic acid encoding a protein having LPA acyltransferase activity, wherein said protein comprises:
a. a sequence encoding the amino acid sequence set forth in SEQ ID No 1.
b. a sequence that is at least 90%, 95%, 97%, 98%, 99%, identical to the sequence in a., wherein said sequence codes for a protein having acyltransferase activity.
c. a fragment of the sequence in a or b, wherein said fragment contains at least 350 amino acids and codes for a protein having acyltransferase activity.

2. The isolated nucleic acid of claim 1 where said nucleic acid is isolated from Litchi sinensis.

3. The isolated acid nucleic of claim 2, coding for a protein comprising SEQ ID NO 2.

4. The nucleic acid of claim 1, comprising a sequence that is greater than 80%, identical to SEQ ID No 3 or SEQ ID No 4.

5. A chimeric gene comprising a nucleic acid sequence of claim 1, linked to suitable regulatory sequences for functional expression.

6. The chimeric gene of claim 5 wherein said regulatory sequence comprises a seed specific promoter.

7. The chimeric gene of claim 6, comprising the Brassica napus napin promoter.

8. A plant transformation vector comprising a nucleic acid sequence of claim 1.

9. A plant transformation vector comprising a chimeric gene of claim 1.

10. A method for expressing a LPA acyltransferase in a plant cell comprising
a. providing a vector of claim 8; and
b. transfecting said plant cell with said vector

11. A plant cell transformed with a vector according to claim 8.

12. The plant cell of claim 11, further expressing a transgene coding for a CFA synthase protein.

13. The plant cell of claim 12, wherein said CFA synthase is selected from the group consisting of SEQ ID No 6, SEQ ID No 7, SEQ ID No 8 and SEQ ID No 10.

14. A method for producing a fertile plant expressing a LPA acyltransferase comprising the steps of
a. providing a vector according to claim 8
b. transfecting a suitable plant tissue with the vector
c. regenerating a fertile plant expressing a LPA acyltransferase.

15. A plant comprising a cell transformed with a vector according to claim 8.

16. The plant of claim 15, further expressing a transgene coding for a CFA synthase protein.

17. The plant of claim 16, wherein said CFA synthase is selected from the group consisting of SEQ ID No 6 and SEQ ID No 7, SEQ ID No 8 and SEQ ID No 10.

18. The plant of claim 15, wherein said plant is an oil producing crop plant.

19. The plant of claim 18 being from the Brassica napus species.

20. Oil from the transgenic plant of claim 15.

21. An isolated protein having LPA acyltransferase activity, comprising;
a. the amino acid sequence set forth in SEQ ID No 1.
b. a sequence that is at least 90%, 95%, 97%, 98%, 99%, identical to the sequence in a., wherein said sequence has acyltransferase activity.
c. a fragment of the sequence in a or b, wherein said fragment contains at least 350 amino acids and has acyltransferase activity.

22. The isolated protein of claim 21, wherein said protein is isolated from Litchi sinensis.

23. The isolated protein of claim 22, comprising the amino acid sequence set forth in SEQ ID No 2.


SEQUENCE LISTING (10 sequences)

SEQ ID NO: 1 - protein, 387 aa, artificial
  synthetic LPAAT derived from litchi
MAVAAAAVVVPLGLLFFISGLVVNLFQAVCFVTIRPLSKNLYRRINRVLAELLWLELVWIVDWWAGVKIKLYTDSETFHLMGKEHALVVSNHRSDIDWLVGWVLAQRSGCLGSALAVMKKSSKLLPVIGWSMWFSEYLFLERNWAKDESTLRSGLQRLKDFPKPFWLALFVEGTRFTQAKLLAAQEYATSTGLPIPRNVLIPRTKGFVASVSHMRSFVPAIYDTTVAIPKSSPAPTMLRLFKGQSSVVHVHIKRCLMKDLPETDDTVAQWCRDIFVAKDELLDKHNVEDTFNDGELQDTSRPVKSLLVVTSWACLVVLGALKFLQWSSLLSSWKGIAFSALGLGVVTVLMHILILFSQSERSTPAKVAPAKPKNNGEPSTRSPAHPK

SEQ ID NO: 2 - protein, 387 aa, artificial
  synthetic LPAAT derived from litchi
MAVAAAAVVVPLGLLFFISGLVVNLFQAVCFVTIRPLSKNLYRRINRVLAELLWLELVWIVDWWAGVKIKLYTDSETFHLMGKEHALVVSNHRSDIDWLVGWVLAQRSGCLGSALAVMKKSSKLLPVIGWSMWFSEYLFLERNWAKDESTLRSGLQRLKDFPKPFWLALFVEGTRFTQAKLLAAQEYATSTGLPIPRNVLIPRTKGFVASVSHMRSFVPAIYDTTVAIPKSSPAPTMLRLFKGQSSVVHVHIKRCLMKDLPETDDTVAQWCRDIFVAKDELLDKHNVEDTFNDGELQDTSRPVKSLLVVTSWACLVVLGALKFLQWSSLLSSWKGIAFSALGLGVVTVLMHILILFSQSERSTPAKVAPAKPKNNGEPSTRSQDKQK

SEQ ID NO: 3 - DNA, 1164 bp, artificial
  synthetic LPAAT derived from litchi
atggcggtagcagcagcagctgtggtcgtccctttaggtcttctcttcttcatttccggcctcgtcgtcaatctctttcaggcagtttgctttgttactattcgacctctgtcgaagaatctgtacaggaggatcaatagagtgctggccgaattgttgtggctggagctcgtgtggatcgttgattggtgggcgggagtcaagatcaaactttacacagatagtgaaacatttcatttaatgggtaaagaacatgcacttgtggtttctaatcatagaagtgacattgattggcttgttggatgggtcttggctcagcggtctggttgtcttggcagtgcgttagctgtaatgaagaaatcatcaaaattgcttccagtcataggttggtcaatgtggttttctgagtatctttttctggaaagaaactgggccaaggacgaaagtactttaaggtcaggtcttcaacggctaaaggacttccctaagcccttttggttggctctatttgtagaaggaactcgcttcacacaggcaaagcttttagcagctcaggaatatgcaacttctacaggcttgcctattcctagaaatgttctgattcctcgtaccaaggggtttgttgcttcagtaagtcatatgcgctcatttgtcccagccatatatgatacaacagtagcaatccctaaaagttcacctgcaccaacaatgctcagactcttcaaggggcaatcttctgtggtgcatgtgcacattaagcgatgtttgatgaaggacttgcctgaaacagatgatactgttgcacagtggtgtagagatatttttgtggccaaggatgaacttttagacaaacacaacgtagaggacaccttcaatgatggagagctgcaagatactagtcggccagtaaagtcccttttggttgttacctcttgggcatgtttggttgttttgggggccctgaagtttctccaatggtcctcacttttatcctcatggaagggtattgcattctcagcattaggtttgggtgttgttacggttcttatgcacatcttgatactcttttctcaatcggagcgttctacccctgccaaggtcgccccagcaaagccaaagaacaatggagaaccttcgacgagaagtccagcccacccaaagtag

SEQ ID NO: 4 - DNA, 1164 bp, artificial
  synthetic LPAAT derived from litchi
atggcggtagcagcagcagctgtggtcgtccctttaggtcttctcttcttcatttccggcctcgtcgtcaatctctttcaggcagtttgctttgttactattcgacctctgtcgaagaatctgtacaggaggatcaatagagtgctggccgaattgttgtggctggagctcgtgtggatcgttgattggtgggcgggagtcaagatcaaactttacacagatagtgaaacatttcatttaatgggtaaagaacatgcacttgtggtttctaatcatagaagtgacattgattggcttgttggatgggtcttggctcagcggtctggttgtcttggcagtgcgttagctgtaatgaagaaatcatcaaaattgcttccagtcataggttggtcaatgtggttttctgagtatctttttctggaaagaaactgggccaaggacgaaagtactttaaggtcaggtcttcaacggctaaaggacttccctaagcccttttggttggctctatttgtagaaggaactcgcttcacacaggcaaagcttttagcagctcaggaatatgcaacttctacaggcttgcctattcctagaaatgttctgattcctcgtaccaaggggtttgttgcttcagtaagtcatatgcgctcatttgtcccagccatatatgatacaacagtagcaatccctaaaagttcacctgcaccaacaatgctcagactcttcaaggggcaatcttctgtggtgcatgtgcacattaagcgatgtttgatgaaggacttgcctgaaacagatgatactgttgcacagtggtgtagagatatttttgtggccaaggatgaacttttagacaaacacaacgtagaggacaccttcaatgatggagagctgcaagatactagtcggccagtaaagtcccttttggttgttacctcttgggcatgtttggttgttttgggggccctgaagtttctccaatggtcctcacttttatcctcatggaagggtattgcattctcagcattaggtttgggtgttgttacggttcttatgcacatcttgatactcttttctcaatcggagcgttctacccctgccaaggtcgccccagcaaagccaaagaacaatggagaaccttcgacgagaagtcaagacaaacaaaagtag

SEQ ID NO: 5 - protein, 390 aa, Brassica napus
MAMAAAVIVPLGILFFISGLVVNLLQAVCYVLVRPLSKNTYRKINRVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESEDEIAQWCRDQFVTKDALLDKHIAADTFAGQKEQNIGRPIKSLAVVLSWACLLTLGAMKFLHWSNLFSSWKGIALSALGLGIITLCMQILIRSSQSERSTPAKVAPAKPKDNHQSGPSSQTEVEEKQK

SEQ ID NO: 6 - protein, 459 aa, Litchi sinensis
MIAAHSLLGKSCAILSKPRHMAPSMMETGARLFVTRFLGQYIIAGRLILMEDGGAVFKFEGTRKNCCLETILRIHNPEFYWKVMTEADLGFADSYINGDFSFVDKDKGLLNLFLIFIANSHLDPLRAKLNQKRGWWSPMFFTAGIASAKYFFRHILNRNTLTQARRNISRHYDLSNDLFALFLDESMTYSCAVFKSEDEDLKVAQMRKLSLLIEKARISKEHEVLDIGCGWGSLAIEAVKRTGCKYTGISLSVEQIKYADIKVKEAGLQDQVTLIHCDYRQLPKTNKYDRIISVGMIDSVGHEYFEEFFGCCESLLAEDGLFVLQYISVPDQHYNEHRLSPGFIKEYIFPGGCLPSLNRITTAMTSASRLCVEHVENIGIHYHQTLRWWRKNLLERQSEVLALGFDEKFIRTWEYYFDYCAAGFKSRTLGDYQIVFSRPGNVAAFTNPYKGFPSTDSVM

SEQ ID NO: 7 - protein, 873 aa, Litchi sinensis
MSEKMKMAVVGGGMSGVVAAHVLAKAGVEVVVYEKKNYLGGQAKIVTFDGVDIDLGCMFFNGMTCSNMMEMSESLGLEMEIVEMSFSVSLDQGQGCEWGTRNGLTSLFAQKKNVFNPYFWQMLREITKFKKDAISYFEILENNPDINYDETLEQFVKSHNYSELFQKAYLIPVCCIIWSCPAERVMSFPILYVFSVYRNHRLFQLLGGPQWLTTRWRSDNYYKRVKEQLENWGCQIRTSSEVYSVSPVDEGCAVICDDGSKEFYNGCIMAVRAPDALKMLGSQATWDELRILGAFQYVCSDIFLHRDKNFMPRNQAAWSACNFMRITGSKTVCLTYWLNVLQDIGEKSSPFLVTLNPEHTPEQTLLKWSTGHPVPSVAASKASRELDLIQGKRGIWFCGAYQGYGFHEDQLKSGLIAAHSLLGKSCAILSKPRHMAPSMMETGARLFVTRFLGQYIIAGRLILMEDGGAVFKFEGTRKNCCLETILRIHNPEFYWKVMTEADLGFADSYINGDFSFVDKDKGLLNLFLIFIANSHLDPLRAKLNQKRGWWSPMFFTAGIASAKYFFRHILNRNTLTQARRNISRHYDLSNDLFALFLDESMTYSCAVFKSEDEDLKVAQMRKLSLLIEKARISKEHEVLDIGCGWGSLAIEAVKRTGCKYTGISLSVEQIKYADIKVKEAGLQDQVTLIHCDYRQLPKTNKYDRIISVGMIDSVGHEYFEEFFGCCESLLAEDGLFVLQYISVPDQHYNEHRLSPGFIKEYIFPGGCLPSLNRITTAMTSASRLCVEHVENIGIHYHQTLRWWRKNLLERQSEVLALGFDEKFIRTWEYYFDYCAAGFKSRTLGDYQIVFSRPGNVAAFTNPYKGFPSTDSVM

SEQ ID NO: 8 - protein, 864 aa, Sterculia foetida
MGVAVIGGGIQGLVSAYVLAKAGVNVVVYEKEEQVGGHAKTVSFDAVDLDLGLLFLNPARYPTMLELFDSLEVDVEATDVSFSVSHDKGNGYEWCSQYGFSNFLAHKKKMLNPYNWQDLRETIKFGNDVNSYLESLEKNPDIDRNETLGHFVGSKGYSENFLNTYLAPICGSMWSCSKEEVMSFSAYSILSFCRTYHLYQLFGNPQWLTIKRHSYLVKKVRDILESRGCQFKLGCEVLSVLPADDGSSIVFGDGFQETYNGCIMAVNAPTALKILGNQATFEEMRVLGAFQYASSDIYLHRDSNLMPTNRSGWSALNFLRSRENKASLTYWLNVLQNVGKTSQPFFVTLNPDRIPDKILLKWSTGRPIPSVAASKASLELDQIQGKRGIWFCGYDFHEDELKAGMDAAHRILGKHFSVLHSPRQMSPSFMETTARLLVTKFFHQYIQVGCVIIIEEGGRVYTFKGSMENCSLKTALKVHNPQFYWRIMKEADIGLADAYIQGDFSFVDKDDGLLNLFRILIANKELNSASGQNKRRTWLSPALFTAGISSAKYFLKHYMRQNTVTQARRNISRHYDLSNELFTLYLGEMMQYSSGIFKTGEEHLDVAQRRKISSLIDKSRIEKWHEVLDIGCGWGSLAMEVVKRTGCKYTGITLSEQQLKYAEEKVKEAGLQGNIKFLLCDYRQLPKTFKYDRIISVEMVEHVGEEYIEEFFRCCDSLLAENGLFVLQFISIPEILSKEIQQTAGFLKEYIFPGGTLLSLDRTLSAMAAASRFSVEHVENIGISYYHTLRWWRKNFLANESKVLALGFDEKFMRTWEYYFDYCAAGFKTGTLIDYQVVFSRAGNFAALGDPYIGFPSAYSYSDN

SEQ ID NO: 9 - DNA, 1173 bp, Brassica napus
atggcgatggcagcagcagtgattgtgcctttggggattctcttcttcatttctggcctcgttgtcaatctccttcaggcagtttgctatgtcctcgttcgacctctgtctaagaacacatacagaaagatcaaccgggtggttgcagaaaccttgtggttggagcttgtctggatcgttgactggtgggctggagtcaagatccaagtctttgctgatgatgagacctttaatcgaatgggcaaagaacatgctcttgtcgtttgtaatcaccgaagtgatattgattggctcgtgggatggattctcgctcagaggtcaggttgcctaggaagcgcattagctgtgatgaagaagtcttccaaatttctcccagtcataggctggtcaatgtggttctccgagtatctgtttcttgaaagaaattgggcaaaggatgaaagcactttaaagtcaggtcttcaacgcttgaacgacttcccacggcctttctggctagctctttttgtggagggaacccgtttcacagaggcaaaacttaaagcagcacaagagtacgcagcctcctctgagttgcctgtccctcgaaatgtgttgattcctcgcaccaaaggatttgtgtcagctgttagtaacatgcgttcatttgtgccagccatatatgatatgaccgtggctattccaaaaacttctccacccccaacgatgctaagactattcaaaggacaaccttctgtggtgcatgttcacatcaagtgtcactcgatgaaagacttgcctgaatcagaagacgaaattgcacagtggtgcagagatcagtttgtgactaaggatgcactgttagacaaacacatagctgcagacactttcgccggtcagaaagaacagaacattggccgtcccataaagtctcttgcagtggttctgtcatgggcatgtctactaactcttggagcaatgaagttcttacactggtcaaatctcttttcctcgtggaaaggcatcgcattatcagcgcttggtctaggcatcatcactctctgtatgcagatcttgatccgctcctctcagtcggagcgttcaacacctgccaaagtcgctccagccaagccaaaggacaatcaccagtcaggaccatcctcccaaacagaagtggaggagaagcagaagtaa

SEQ ID NO: 10 - protein, 864 aa, Sterculia foetida
MGVAVIGGGIQGLVSAYVLAKAGVNVVVYEKEEQVGGHAKTVSFDAVDLDLGLLFLNPARYPTMLELFDSLEVDVEATDVSFSVSHDKGNGYEWCSQYGFSNFLAHKKKMLNPYNWQDLRETIKFGNDVNSYLESLEKNPDIDRNETLGHFVGSKGYSENFLNTYLAPICGSMWSCSKEEVMSFSAYSILSFCRTYHLYQLFGNPQWLTIKRHSYLVKKVRDILESRGCQFKLGCEVLSVLPADDGSSIVFGDGFQETYNGCIMAVNAPTALKILGNQATFEEMRVLGAFQYASSDIYLHRDSNLMPTNRSGWSALNFLRSRENKASLTYWLNVLQNVGKTSQPFFVTLNPDRIPDKILLKWSTGRPIPSVAASKASLELDQIQGKRGIWFCGYDFHEDELKAGMDAAHRILGKHFSVLLSPRQMSPSFMETTARLLVTKFFHQYIQVGCVIIIEEGGRVYTFKGSMENCSLKTALKVHNPQFYWRIMKEADIGLADAYIQGDFSFVDKDDGLLNLFRILIANKELNSASGQNKRRTWLSPALFTAGISSAKYFLKHYMRQNTVTQARRNISRHYDLSNELFTLYLGEMMQYSSGIFKTGEEHLDVAQRRKISSLIDKSRIEKWHEVLDIGCGWGSLAMEVVKRTGCKYTGITLSEQQLKYAEEKVKEAGLQGNIKFLLCDYRQLPKTFKYDRIISVEMVDMVGEEYIEEFFRCCDSLLAENGLFVLQFISIPEILSKEIQQTAGFLKEYIFPGGTLLSLDRTLSAMAAASRFSVEHVENIGISYYHTLRWWRKNFLANESKVLALGFDEKFMRTWEYYFDYCAAGFKTGTLIDYQVVFSRAGNFAALGDPYIGFPSAYSYSDN